(19) United States
(12) Patent Application Publication
(10) Pub. No.: US 2013/0253055 A1
(43) Pub. Date: Sep. 26, 2013

(54) POWDEROUS FORMULATIONS OF FAT-SOLUBLE ACTIVE INGREDIENTS

(71) Applicant: DSM IP ASSETS B.V., Heerlen (NL)

(72) Inventors: Elger FUNDA, Grenzach-Wyhlen (DE); Torsten HUBER, Magden (CH)

(73) Assignee: DSM IP ASSETS B.V., Heerlen (NL)

(21) Appl. No.: 13/887,608

(22) Filed: May 6, 2013

Related U.S. Application Data
(63) Continuation of application No. 13/294,676, filed on Nov. 11, 2011, now abandoned.

(30) Foreign Application Priority Data
Mar. 4, 2003 (EP) 03.007677.2

(51) Int. Cl. A23J 3/14; A61K 8/64; A61K 47/42

(52) U.S. Cl. CPC A23J 3/14; A61K 47/42; A61K 8/64
USPC 514/529; 426/656; 426/73; 426/72; 426/590; 426/52; 514/725; 435/68.1

(57) ABSTRACT

Stable powderous formulations containing a fat-soluble active ingredient, e.g., vitamin A, in a matrix of a native lupin protein composition are disclosed.

The present invention is concerned with novel stable powderous formulations comprising a fat-soluble active ingredient, and a process for their preparation. The novel compositions of this invention can be used as additives for food, beverages, animal feeds, cosmetics or drugs to incorporate said fat-soluble ingredients into such application forms.

More specifically, the present invention is concerned with stable powderous formulations comprising a fat-soluble active ingredient in a matrix of a native lupin protein composition.

As used herein, the term “native lupin protein” denotes a lupin protein as it is found in natural products such as lupin seeds and has not been modified by hydrolysis. However, the term “native lupin protein” is understood to include lupin proteins which have undergone post-isoelectric precipitation and are generally known as “restructured” proteins, see international patent application WO 99/11143 and references contained therein.

The term “native lupin protein composition” denotes any composition comprising native lupin protein as obtainable from natural lupin protein sources. Examples of such native lupin protein compositions are lupin protein concentrates, which have a protein content of 60% up to 90% by weight (hereinafter : wt.-%), generally from 50-96 wt-%, typically about 65-70 wt.-% of protein; and lupin protein isolates, which term is generally used in the art to define protein preparations containing more than about 90 wt.-% of protein. The residual constituents (4-50 wt.-%) of such concentrates and isolates are, besides water and oil, primarily plant fibers.

For the purpose of the present invention, lupin concentrates having a protein content of about 60-90 wt.-%, isolates having a protein content of more than 90 wt.-%, and flours having a protein content of about 40-60 wt.-%, are preferred. As a source for the protein compositions all known lupin varieties, such as Lupine Angustifolius, Lupine Albus oder Lupine Luteus can be used. However, protein compositions derived from Lupine Angustifolius and Lupine Albus are preferred.

The term “fat-soluble active ingredient” as used herein denotes any physiologically active ingredient that is soluble in lipids and insoluble or sparingly soluble in water. Examples of such fat-soluble active ingredients are fat-soluble vitamins, viz., vitamin A, D, E and K and derivatives thereof such as vitamin A esters, e.g. vitamin A acetate and palmiate, and vitamin E esters, e.g. tocopherol acetate; carotenoids and carotinoid derivatives, e.g., are α- or β-carotene, 8′-apo-β-carotenal, 8′-apo-β-carotenoic acid esters such as the ethyl ester, canthaxanthin, astaxanthin, astaxanthin esters, lycopene, lutein, zeaxanthin or crocetin and their derivatives; polyunsaturated fatty acids, e.g. eicosapentaenoic acid, docosahexaenoic acid, arachidonic acid and and γ-linolenic acid and/or ethylester. The fat-soluble active ingredient may be present in the formulation in an amount of from about 0.1 wt.-% to about 80 wt.-%, especially from about 0.5 wt.-% to about 60 wt.-%, based on the total weight of the composition.

In a preferred aspect of the invention, the novel formulations additionally contain a reducing sugar, e.g. glucose, fructose, or xylose in an amount of from about 0.1 wt.-% to about 70 wt.-%, especially from about 1.0 to about 10 wt.-%, based on the total weight of the composition.

Such formulations can be submitted to heat-treatment to cause cross-linking of the sugar with the protein in a Maillard type reaction. Crosslinking can be also achieved by treatment with enzymes like transglutaminase in a manner know per se, see, e.g., U.S. Pat. No. 5,156,956. The cross-linked formulations have been found to exhibit increased stability.

In accordance with the invention, the novel formulations can be obtained by a process which comprises preparing an aqueous emulsion of the fat-soluble active ingredient and the native lupin protein composition, if desired, adding a reducing sugar, converting the emulsion into a dry powder and, if a reducing sugar was added, submitting the dry powder to cross-linking the sugar with the protein by heat treatment or by treatment with a cross-linking enzyme.

Suitably, in a first step of the process of the invention, the protein composition is dispersed in water. Thereafter, the fat-soluble active ingredient is emulsified, suitably in liquid state, i.e. with adequate warming and/or as a solution in an appropriate solvent, into the aqueous dispersion of the protein. Alternatively a suspension of the solid active may be produced by appropriate procedures like milling. The emulsion is then, optionally after removal of excess solvent, spray-dried. The spray-drying can effected be using conventional technology of spray-drying, spray drying in combination with fluidized-bed granulation (the latter technique commonly known as fluidized spray drying or FSD), or by a powder-catch technique where sprayed emulsion droplets are caught in a bed of an absorbant such as starch or calcium silicate and subsequently dried.

In still another aspect of the invention, the novel formulations may additionally contain other proteins or hydrolyzed proteins that act as protective colloids, e.g. soy proteins or, hydrolyzed soy proteins. Such additional proteins may be present in the formulations of the invention in an amount of from 10-50 wt.-% based on the total amount of protein in the formulation.

Finally, in a still further aspect, the present invention is concerned with food, beverages, animal feeds, cosmetics and drugs which comprise the novel formulations of the present invention.

The novel formulations of this invention may further contain adjuvants and/or excipients such as one or more of a mono- di-, oligo- or polysaccharide, a triglyceride, a water-soluble antioxidant, a fat-soluble antioxidant, silicic acid, Ca-silicate, Ca-carbonate and water.

Examples of mono- and disaccharides which may be present in the formulations of the present invention are saccharose, invert sugar, glucose, fructose, lactose and maltose. Examples of oligo- or polysaccharides which may be present in the compositions of the present invention are starch, modified starch and starch hydrolysates, such as dextrins and maltodextrins, especially such in the range of 5-65 dextrose equivalents (hereinafter: DE) and glucose syrup, especially such in the range of 20-95 DE. The term “dextrose equivalent” (DE) denotes the degree of hydrolysation and is measure for the amount of reducing sugar calculated as D-glucose based on dry weight. Native starch has DE close to 0 while glucose has a DE=100.

The triglyceride is suitably a vegetable oil or fat, such as corn oil, sunflower oil, soybean oil, safflower oil, rape seed oil, arachis oil, palm oil, palm kernel oil, cotton seed oil or cocos oil.

The water-soluble antioxidant may be ascorbic acid and salts thereof, e.g., sodium ascorbate, and the like. The fat-soluble antioxidant may be a tocopherol, e.g., dl-α-tocopherol (i.e., synthetic tocopherol), d-α-tocopherol (i.e., natural tocopherol), β- and γ-tocopherol and mixtures thereof; ascorbic acid esters of fatty acids such as ascorbyl palmitate or stearate; butyl hydroxy toluene (BHT); butyl hydroxy anisol (BHA); propyl gallate; or t-butyl hydroxy quinoline; or 6-ethoxy-1,2-dihydroxy-2,2,4-trimethylquinoline (EMQ).

The following Examples illustrate the invention further.

### EXAMPLE 1

### Preparation of a Powderous Vitamin A Formulation:

62.4 g of lupin protein isolate from Lup. Angustifolius (protein content 96.2%) and 10.9 g of glycerol were added to 230 ml of water. The mixture was warmed to 60° C. until dissolution occurred. To this solution, 12.3 g of fructose were added and the pH of the solution was adjusted to 6.5±0.2. Thereafter, 49.3 g of vitamin A acetate (2.1×10⁶ IE vitamin A/g stabilized with Ethoxyquin) were emulsified into the matrix solution whereupon the mixture was stirred for 60 minutes at 60° C. The inner phase of the emulsion then exhibited a mean particle size of about 580 nm. The emulsion was then diluted with ca. 25 ml of water and about 300 g of the emulsion was sprayed in a spraying pan in a bed of Ca-silicate at about 5° C. by means of a rotating spraying nozzle. The so-obtained beadlets were separated from excess Ca-silicate by sieving and dried. There were obtained ca. 100 g of dry powder having a vitamin A content of ca. 850′000 IEA/g.

### EXAMPLE 2

### Thermal Cross-Linking:

The vitamin A dry powder obtained in Example 1 is stirred at a temperature of 135° C. for 35 minutes. The so-obtained product was insoluble in hot water and had a vitamin A content of ca. 570′000 IEA/g.

### EXAMPLE 3

### Preparation of an Ethyl Apo-Carotenoate Dry Powder:

a) 16 g of lupin protein isolate from Lup. Angustifolius (protein content 96.2%) were dissolved in 130 ml of water at 50° C. To this solution, 1.6 g of ascorbylpalmitate were added and the pH of the solution was adjusted to 7.5±0.2 by the addition of 20 wt.-% sodium hydroxide solution.

b) 9 g of ethyl β-apo-8′-carotenoate, 5.5 g of corn oil and 0.6 g of Ethoxyquin were dissolved in 50 ml of chloroform.
c) The ethyl β-apo-8′-carotenoate solution obtained in paragraph b) was emulsified during 30 minutes at 45° C. into the solution obtained in paragraph a). The inner phase of the emulsion then exhibited a mean particle size of about 280 nm. The chloroform was evaporated at 50° C. under reduced pressure and the emulsion was spray-dried in analogy to the procedure of Example 1 in a bed of starch. There were obtained 42 g of dry powder having an ethyl β-apo-8′-carotenoate content of 11.4 wt.- %.

1. Stable powderous formulations comprising a fat-soluble active ingredient in a matrix of a native lupin protein composition wherein the protein is cross-linked.

2. Formulations according to claim 1, wherein the lupin protein composition is a lupin protein isolate having a protein content of more than 90 wt.-%.

3. Formulations according to claim 1, wherein the lupin protein composition is a lupin protein concentrate having a protein content of about 60-90 wt.-%.

4. Formulations according to claim 1, wherein the lupin protein composition is a lupin protein flour having a protein content of about 40-60 wt.-%.

5. Formulations according to claim 1, wherein the native lupin protein composition is selected from the group consisting of a lupin protein isolate having a protein content of more than 90 wt.-%, a lupin protein concentrate having a protein content of about 60-90 wt.-%, a lupin protein flour having a protein content of about 40-60 wt.-%, and mixtures of any of the foregoing.

6. Formulations according to claim 1, wherein the fat-soluble active ingredient is vitamin A, D, E or K, or a carotenoid, or a polyunsaturated fatty acid, or esters thereof, or mixtures thereof.

7. Formulations according to claim 1, wherein the fat-soluble active ingredient is a plant or animal oil or fat, particularly sunflower oil, palm oil or corn oil.

8. Formulations according to claim 1, comprising additionally a reducing sugar, particularly glucose, fructose, or xylose.

9. Formulations according to claim 1, wherein the protein is cross-linked.

10. Food, beverages, animal feeds, cosmetics or drugs comprising a formulation according to claim 1.

11. A process for the preparation of a formulation comprising preparing an aqueous emulsion of a fat-soluble active ingredient and a native lupin protein composition.

12. A process according to claim 11, wherein a reducing sugar is added and the composition is submitted to cross-linking by heating.

13. A process according to claim 11, wherein the composition is submitted to cross-linking by treatment with a cross-linking enzyme, particularly transglutaminase.

14. A process for the preparation of a formulation comprising preparing an aqueous emulsion of a fat-soluble active ingredient and a native lupin protein composition, adding a reducing sugar, converting the emulsion into a dry powder, and if appropriate, submitting the dry powder to cross-linking the protein by heat treatment or by treatment with a cross-linking enzyme.

15. Formulations according to claim 7, wherein the plant oil is selected from the group consisting of sunflower oil, palm oil and corn oil.

16. Formulations according to claim 8, wherein the reducing sugar is selected from the group consisting of glucose, fructose, and xylose.

17. A process according to claim 13, wherein the cross-linking enzyme is transglutaminase.

18. A process according to claim 14, further comprising submitting the dry powder to heat treatment or treatment with a cross-linking enzyme, to cross-link the protein of the dry powder.
